(19) United States
(12) Patent Application Publication
(10) Pub. No.: US 2016/0015434 A1
(43) Pub. Date: Jan. 21, 2016

(54) Cap Device, Particularly for Closing A Burr Hole

(71) Applicant: Universitat Bern, Bern (CH)

(72) Inventors: Lennard STIEGLITZ, Zurich (CH); Andreas RAABE, Muri (CH)

(73) Assignee: UNIVERSITAT BERN, Bern (CH)

(21) Appl. No.: 14/331,234

(22) Filed: Jul. 15, 2014

(51) Int. Cl. A61B 17/80

(52) U.S. Cl. CPC A61B 17/8061; A61B 17/8085

(57) ABSTRACT

The present invention relates to a cap device for closing a hole, particularly a burr hole, in a bone, particularly a skull, of a human or an animal, comprising a frame member for fastening the cap device to the bone, and a cap member for covering the hole. According to the invention, the cap member is integrally connected to the frame member, so that the cap member can be bent away from the frame member, particularly for allowing passage of a catheter through said hole.

### BACKGROUND

The present invention relates to a cap device.

Chronic subdural hematomas are among the most frequent lesions admitted for neurosurgical treatment. The incidence is between 3.4/100.000 in young patients and 8-58/10.000 in the elderly (over 65 years). Especially in old patients these hematomas typically occur days or weeks after a minor head trauma and tend to grow slowly with time. As reasons for the growth repeated fresh bleedings into the hematoma and an aseptic inflammatory process are discussed. Treatment is based on a surgical drainage of the hematoma. This can be achieved either by craniotomy or using burr holes. A burr hole is a hole that is surgically formed in the skull, which is used to pass a drainage catheter so as to allow for a cerebrospinal fluid drainage or evacuation of chronic blood.

For evacuation of the hematoma usually a drain is implanted in the hematoma cavity (subdural space) and externalized through the skin.

For cosmetic reasons the anterior burr hole may be closed. The posterior burr hole usually remains open (skin closed above) as the drain has to pass it.

Despite good recovery from the hematoma and the evacuation patients frequently suffer from skin retractions into the burr hole, which is visible and palpable from outside.

Therefore, in view of the foregoing, there is a need to provide for a cap device that allows a drain to pass (e.g. for several hours or days) and closes the burr hole neatly, particularly without need for a separate surgical intervention when the drain is removed.

### SUMMARY OF THE INVENTION

According to an aspect of the present invention, a cap device for closing a hole (particularly a burr hole) in a bone (particularly a skull) of a human or an animal is disclosed, wherein the cap device comprises a frame member for fastening the cap device to the bone, and a cap member for covering the hole, wherein the cap member is integrally connected to the frame member, so that the cap member can be bent (or pivoted) away from the frame member for opening the cap device, particularly for allowing passage of a catheter through said hole and particularly through the cap device.

Thus, the cap device according to the invention allows closing of the (burr) hole despite a drain that is implanted in the hematoma cavity through the (burr) hole. When the drain is removed, the cap member closes the (burr) hole and stabilizes the skin on top of the cap member so that skin retractions into the (burr) hole can be prevented.

Since the cap member being hinged to the frame member is elastically deformable, the cap member closes automatically after removal of the drainage (e.g. catheter).

According to an embodiment of the present invention, the frame member comprises an annular configuration with a gap. Particularly, said gap is delimited by two free ends of the frame member that face each other resulting in a penannular shape of the frame member. This is advantageous, since a catheter can extend through said gap which reduces the height of the cap device when a catheter extends through the cap device and (burr) hole.

Further, according to an embodiment of the present invention, the frame member and the cap member extend in a common plane when the cap member is not bent away from the frame member. Particularly, the cap member and the frame member are formed as plates or comprise a plate-like shape and may be cut, milled or stamped out of a single sheet of a metal.

Further, according to an embodiment of the present invention, the cap member comprises a first portion for closing the hole, wherein the cap member is integrally connected to the frame member via said first portion. Here, closing does not necessarily mean that the hole is fully closed (e.g. hermetic), but that the hole is at least partially covered with the cap member so that the skin on top of the cap member can be supported. Hence, the cap member may comprise one or several recesses.

Furthermore, according to an embodiment of the present invention, the first portion of the cap member or the cap member comprises a meandering shape. This e.g. means that said first portion/cap member comprises a plurality of legs, i.e. a first leg, at least one or several intermediate legs, and a last leg, each extending in or along a common direction, particularly extending parallel to each other, wherein each leg is integrally connected to its neighboring leg(s). Particularly, a first leg of said plurality of legs is integrally connected to the frame member (so that an integral hinge is formed), particularly at a region facing said gap of the frame member. Further, said last leg is particularly integrally connected to said second portion of the cap member or may form said second portion itself (see below). Further, said intermediate legs each comprise two ends, wherein each end is integrally connected via a connection portion to exactly one neighboring (e.g. parallel) leg. Particularly, said connection portions each extend along the frame member, when the cap member is not bent/pivoted away from the frame member. Also, each of said connection portions particularly extends at an angle to the leg to which it is integrally connected.

Further, according to an embodiment of the present invention, the frame member surrounds at least the first portion of the cap member in a penannular fashion when the plate member is not bent away from the frame member, but resides in its initial position, in which the cap member (and also the cap device) comprises a flat configuration.

Furthermore, according to an embodiment of the present invention, the cap member comprises a second portion connected (particularly integrally) to the first portion, wherein the second portion is arranged in said gap of the frame member, when the cap member is not bent away from the frame member. Particularly, the second portion fills the gap despite a play between said free ends of the frame member and the second portion of the cap member.

Further, according to an embodiment of the present invention, when the cap member is bent away from the frame member, the cap device provides a restoring force that tends to move the cap member back into its initial (flat) position, particularly such that a catheter can be clamped between the cap member and the bone (e.g. skull), particularly between the second portion and the bone.

Furthermore, according to an embodiment of the present invention, the frame member comprises through-holes for fastening the frame member to the skull. Said screw holes are particularly designed for receiving screws by means of which the frame member (and thus the cap device) is particularly fastened to the bone/skull.

Further, according to an embodiment of the present invention, the frame member comprises a penannular portion delimiting said gap as well as fastening portions protruding from the annular portion, wherein each through-hole is at least partially formed in one of the fastening portions.

Further, according to an embodiment of the present invention, the frame member and the cap member are formed out of a metal or comprise a metal, particularly titanium (Ti) or a titanium alloy.

### BRIEF DESCRIPTION OF THE DRAWINGS

The embodiments will be readily understood by the following detailed description in conjunction with the accompanying drawings, wherein:

FIG. 1 shows a perspective view of a cap device according to the invention in an initial state;

FIG. 2 shows a perspective view of the cap device of FIG. 1, wherein the cap member is bent upwards for allowing passage of a catheter; and

FIG. 3 shows an application of a cap device to a burr hole according to the invention.

### DESCRIBED EMBODIMENTS

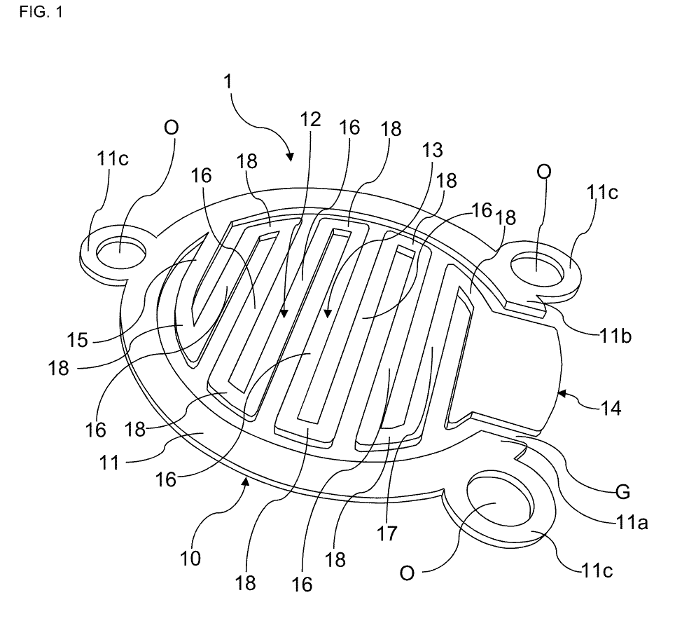

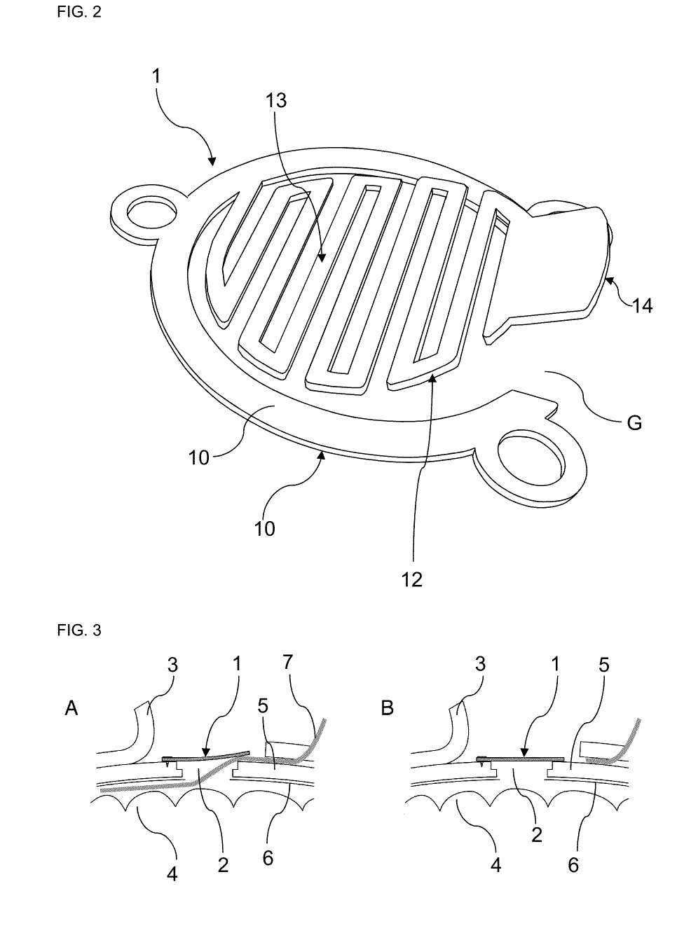

FIG. 1 shows in conjunction with FIG. 2 a cap device (also denoted as burr hole plate) 1 according to the invention, which is designed for closing a burr hole 2 in an (e.g. human) skull 5 (cf. FIG. 3).

The cap device 1 comprises a frame member 10 for fastening the cap device 1 to the bone 5 (e.g. by means of screws) as well as a cap member 12 for covering the hole 2. According to the invention, the cap member 12 is integrally connected/hinged to the frame member 10, so that the cap member 12 can be bent from an initial position, in which the cap member 12 is arranged in the extension plane of the frame member 10 and surrounded by the latter, upwards into an advanced position which allows the passage of a catheter 7 below the cap member 12 into the hole 2 (cf. FIG. 3).

As shown in FIGS. 1 and 2, the frame member 10 comprises a penannular shape. In detail, the frame member 10 comprises a penannular portion 11 to which the cap member 12 is integrally hinged as well as fastening portions 11c protruding outwardly from the penannular portion 13, namely from a region of the penannular portion 13/frame member 10 to which the cap member 12 is hinged as well as on both sides of the gap G that is delimited by two free ends 11a, 11b of the annular portion 11/frame member 10, which free ends 11a, 11b face each other. The fastening portions 11c each comprise a through-opening O through which a screw can be passed by means of which the frame member 10 is fastened to a circumferential edge region of the bone 5, which edge region delimits said hole 2.

The cap member 12 comprises a first portion 13 for covering the hole 2 via which first portion 13 the cap member 12 is integrally connected to the frame member 10, namely to said penannular portion 13 of the frame member 10. Further, the cap member 12 comprises a second portion 14 connected integrally to the first portion 13, wherein the second portion 14 is arranged in the gap G in the initial position of the cap member 12.

As shown in FIGS. 1 and 2, the first portion 13 or the cap member 12 comprises a meandering shape, i.e., extends in a winding manner back and forth between the frame member 10. In detail, said first portion 13 comprises a plurality of (e.g. longitudinally extending) legs 15, 16, 17 extending particularly parallel to each other, wherein each leg 15, 16, 17 is integrally connected to its neighboring leg(s). A first leg 15 of said plurality of legs is integrally connected to the frame member 10, namely to the penannular portion 13, at a region of said portion 13 that faces said gap G. Further, a last leg 17 of said series of legs is integrally connected to said second portion 14 of the cap member 12 (or may form said second portion 14). Compared to the legs 15, 16, 17, the second portion 14 has a larger width than said legs 15, 16 and 17. The intermediate legs 16, which are arranged between the first and the last leg 15, 17, each comprise two ends, wherein each end is integrally connected via a connection portion 18 to exactly one neighboring leg so that the whole first portion 13 of the cap member 12 forms said winding or meandering structure. Due to this structure, the cap member 12 is flexible and can be elastically deformed, e.g. bent upwards, so that the burr hole 2 is accessible for arranging a catheter 7 therein. Furthermore, the individual connection portions 18 follow the circular course of the penannular portion 11 of the frame member 10 so that the cap member 12 comprises essentially a circular outline (despite the recesses due to the meandering configuration), wherein an e.g. small gap is present between the cap member 12 and the frame member 10.

When the cap member 12 is bent upwards, the cap device 1 provides a restoring force due to the integral connection between the cap member 12 and the frame member 10 as well as the fact that both components are formed out of an elastically deformable material (e.g. a metal like for instance titanium or a titanium alloy). This restoring force tries to move the cap member 12 back into its initial position. Thus, when the catheter 7 is removed, the cap device 1 automatically closes the burr hole 2 by moving the cap member 12 back to its initial position where it is aligned with the frame member 10.

FIG. 3 shows an application of the cap device 1 according to FIGS. 1 and 2 to a burr hole 2 in a skull 5. After forming the burr hole 2 extending through the bone 5 and dura 6 above the cortex 4, the cap device 1 is fastened to the bone/skull 5 by means of screws as described above. The cap member 12 is lifted by bending it upwards so that the second portion 14 moves out of the gap G. This provides space for passing a catheter 7 below the cap member 12 through the burr hole 2 and into the hematoma (left panel A of FIG. 3). Once the catheter 7 is removed, the cap member 12 automatically closes the burr hole 2 (right panel B of FIG. 3). The skin 3 can then be arranged on top of the cap device 1.

For testing purposes a small series of 20 cap devices 1 has been produced and implanted at Berne University Hospital in Switzerland. 8 patients received unilateral evacuation, 6 patients were evacuated bilaterally. 7 were of male, 5 of female gender. 6 presented for clinical follow-up already. Implantation together with a drain in the hematoma cavity was feasible and uneventful in all cases. There were no infections, no damaging of draining catheters and no catheter occlusions by the plates. In this series there was one recurrent hematoma. All patients were satisfied with the cosmetic result postoperatively. There were no cases of skin retraction into the burr hole in this series.

The various aspects, embodiments or features of the described embodiments can be used separately or in any combination.

The foregoing descriptions of specific embodiments of the present invention are presented for purposes of illustration and description. They are not intended to limit the invention to the precise forms disclosed. It will be apparent to those skilled in the art that many modifications and variations of the invention are possible in view of the above teachings.

It is therefore intended that the scope of the invention be defined by the following appended claims and their equivalents.

1. A cap device for closing a burr hole in a skull of a human or an animal, comprising
an annular frame member with a gap for fastening the cap device to the bone, wherein the gap is delimited by two free ends of the frame member, which two ends face each other resulting in a penannular shape of the frame member, and
a cap member for covering the hole,
wherein the cap member is integrally connected to the frame member, so that the cap member can be bent away from the frame member for opening the cap device, wherein the cap member comprises a first portion for closing the hole, wherein the cap member is integrally connected to the frame member via said first portion, and wherein the cap member comprises a second portion connected to the first portion, wherein the second portion is arranged in said gap when the cap member is not bent away from the frame member.

2. (canceled)

3. (canceled)

4. The cap device as claimed in claim 1, wherein the frame member and the cap member extend in a common plane when the cap member is not bent away from the frame member.

5. (canceled)

6. The cap device as claimed in claim 1, wherein the first portion of the cap member comprises a meandering shape.

7. The cap device as claimed in claim 1, wherein the frame member surrounds at least the first portion of the cap member when the cap member is not bent away from the frame member.

8. (canceled)

9. The cap device as claimed in claim 1, wherein when the cap member is bent away from the frame member, the cap device provides a restoring force that tends to move the cap member back into its initial position.

10. The cap device as claimed in claim 1, wherein the frame member comprises through-holes for fastening the frame member to the bone.

11. The cap device according to claim 10, wherein the frame member comprises a penannular portion delimiting said gap as well as fastening portions protruding from the penannular portion, wherein each through-hole is at least partially formed in one of the fastening portions.

12. The cap device as claimed in claim 1, wherein the frame member and the cap member are formed out of a metal or comprise a metal.

13. The cap device as claimed in claim 12, wherein said metal is titanium or a titanium alloy.

14. (canceled)

15. A cap device for closing a burr hole in a skull of a human or an animal, comprising
a frame member for fastening the cap device to the bone, wherein the frame member is c-shaped and comprises a gap delimited by two ends of the frame member, which two ends face each other, and
a cap member for covering the hole,
wherein the cap member is integrally connected to the frame member, so that the cap member can be bent away from the frame member for opening the cap device, wherein the cap member comprises a first portion for closing the hole, wherein the cap member is integrally connected to the frame member via said first portion, and wherein the cap member comprises a second portion connected lo the first portion, wherein ihe second portion is arranged in said gap when the cap member is not bent away from the frame member.

16. A cap device lor closing a burr hole in a skull of a human or an animal, comprising
an annular frame member with a gap for fastening the cap device to the bone, wherein the gap is delimited by two free ends of the frame member, which two ends face each other resulting in a penannular shape of the frame member, and
a cap member for covering the hole,
wherein the cap member is integrally connected to the frame member, so that the cap member can be bent away from the frame member for opening the cap device, wherein the cap member comprises a first portion for closing the hole, wherein the cap member is integrally connected to the frame member via said first portion, and wherein the cap member comprises a second portion connected to the first portion, wherein the second portion is arranged in said gap when the cap member is not bent away from the frame member, and wherein the second portion substantially fills said gap when the cap member is not bent away from the frame member.
